(19) United States
(12) Patent Application Publication
(10) Pub. No.: US 2012/0136288 A1
(43) Pub. Date: May 31, 2012

(54) HEAT MASSAGE GLOVES

(76) Inventor: Deborah Napier, Garland, TX (US)

(21) Appl. No.: 12/955,904

(22) Filed: Nov. 30, 2010

(51) Int. Cl. A61H 1/00; A61F 7/00; A41D 19/00

(52) U.S. Cl. 601/48; 2/159; 607/96

(57) ABSTRACT

A heated massage glove comprising a hand covering to envelope a hand of a user, a set of finger segments to accept the fingers of the user, a set of finger massagers attached within the set of finger segments, where the set of finger massagers vibrate and pulse against the set of fingers, a set of hand massagers attached to a backside of the hand covering, wherein the set of hand massagers vibrate and pulse against a backside of the hand, a heating apparatus housed within the hand covering, where the heating apparatus includes a plurality of wires to heat the hand covering, and a control mechanism attached to the backside of the hand covering wherein the control mechanism controls how intense the set of finger massagers and the set of hand massagers vibrates and pulses and how intense the heating apparatus heats.

### BACKGROUND OF THE INVENTION

1. Field of the Invention

The present invention relates to a massage glove that includes an internal heating apparatus and a vibrating device to provide comfort and relief to a user's hands.

2. Description of Related Art

Many people experience aches and soreness commonly associated with arthritis, carpal tunnel, and stress on a daily basis. The pain may come and go throughout the day or remain a constant nuisance, often interfering with their work performance or other daily activities. Some individuals choose to relieve the aches and pains with oral medications, injections, rubbing ointments or splints. These methods help to mask the pain temporarily but sometimes the pain returns more intense.

Some individuals find relief with the help of massage therapy. By rubbing and heating the sore joints and bones the person's pains are soothed and often relieved. Since massage therapy is expensive for repeated or continuous use, massaging gloves have been developed to assist the aching joints. For example, U.S. Pat. No. 5,601,529 by Matt J. Wollman discloses a Finger Massage Apparatus wherein an adapted glove fits over the thumb and a single finger of the user. The glove sends a current to activate vibrating motors housed within the finger portions to massage the fingers of the user. The Finger Massage Apparatus is useful for individual fingers, but only functions to work on one finger at a time. Alternatively, U.S. Application No. 2004/0087881 by Julia Gross discloses an Electrical Heat and Massage Vibrating Glove wherein a leather glove massages the user's hand around the wrist area and heats the hand for additional relief. The glove is controlled by a turn dial to vary the intensity of the massage. While this glove does combine heat and massage in a glove, the heating element only warms the palm portion of the hand and the massagers only vibrate around the wrist portion. Therefore, relief is not evenly distributed across the entire hand.

It would be beneficial in the art to provide a massage glove that combines a heating apparatus to offer pain relief to arthritis, carpal tunnel and stress sufferers. It would also be desirable in the art to provide a glove that massages more than one portion of the hand.

### SUMMARY OF THE INVENTION

An object of the present invention is to provide a heated massage glove to provide pain relief to the sufferers of arthritis, carpel tunnel and stress.

Another object of the present invention is to provide a heated massage glove that includes a set of finger massagers, a set of hand massagers and a set of wrist massagers.

Another object of the present invention is to provide a heated massage glove that is operated by control mechanism which controls the intensity of the set of finger massagers, the set of hand massagers and the set of wrist massagers.

In view of the foregoing disadvantages inherent in the prior art, the present disclosure provides a heated massage glove comprising a hand covering to envelope a hand of a user, a set of finger segments to accept a set of fingers of the user, a set of finger massagers attached within the set of finger segments, where the set of finger massagers vibrate and pulse against the set of fingers, a set of hand massagers attached to a backside of the hand covering, wherein the set of hand massagers vibrate and pulse against a backside of the hand, a heating apparatus housed within the hand covering, where the heating apparatus includes a plurality of wires to heat the hand covering, and a control mechanism attached to the backside of the hand covering wherein the control mechanism controls how intense the set of finger massagers and the set of hand massagers vibrates and pulses and how intense the heating apparatus heats.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawing, wherein like elements are identified with like symbols, and in which:

FIG. 1 depicts a perspective view of a heated massage glove in accordance with an exemplary embodiment of the present invention.

### DETAILED DESCRIPTION OF THE DRAWINGS

The present invention relates to a massage glove that includes an internal heating apparatus and a vibrating device to provide comfort and relief to a user's hands. The present invention discloses a heated massage glove as a way to alleviate pain and soreness in the user's hands, fingers, knuckles and wrists which is commonly associated with overuse, arthritis carpel tunnel and stress. The heated massage gloves include a pair of form fitted, battery operated gloves that remedy pains by radiating heat and utilizing massage therapy. The deep penetrating heat and multiple vibration controls of the heated massage gloves provide ease and comfort even for those users with deep settled stiffness in the joints. The heated massage gloves are conveniently lightweight, and are appropriate for use at home, work or while traveling.

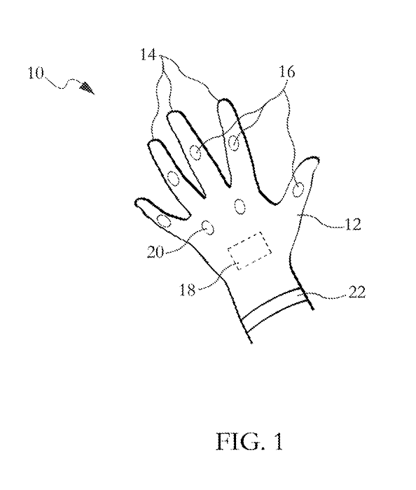

Turning now descriptively to the drawings, referring to FIG. 1, a perspective view of a heated massage glove (10) in accordance with an exemplary embodiment of the present invention. The heated massage glove (10) includes a hand covering (12) or glove which may completely envelope a hand of a user. The hand covering (12) may be form fitted to the user's hand to provide a secure yet comfortable fit. The hand covering (12) may be made from a breathable, comfortable, flexible and soft material such as a mesh material, a neoprene material, a cotton or cotton blend like terrycloth or the like. Alternatively, the hand covering (12) may simply cover a backside of the user's hand with an exposed palm or possibly only a few straps crossing the palm to secure the heated massage glove (10). The heated massage glove (10) may be available as a pair to provide relief to both of the user's hands.

Covering each of the user's fingers is a set of finger segments (14). The set of finger segments (14) may also be well fitted around the user's finger to ensure that the heated massage glove (10) does not slide off during use. Along the set of finger segments (14) may be a set of finger massagers (16) which may be attached at a midpoint of the set of finger segments (14). The midpoint of the set of finger segments (14) may coordinate with a set of finger knuckles of the user. Therefore the set of finger massagers (16) rests above the set of finger knuckles. The set of finger massagers (16) may vibrate or pulse depending on the preference of the user. The vibrations and pulsations may relieve the user of any pains, soreness or aches commonly associated with arthritis, carpal tunnel or stress. By including a finger massager (16) on each of the user's fingers the heated massage glove (10) provides concentrated relief for each individual finger.

Along a backside portion of the hand covering (12) may be a set of hand massagers (20). The set of hand massagers (20) may be situated above a set of hand knuckles of the user. The set of hand massagers (20) may vibrate and pulse similar to how the set of finger massagers (16) operates to relieve aches and pains in the user's knuckles. The set of finger massagers (16) and the set of hand massagers (20) may function independent of each other. Therefore, when the set of finger massagers (16) are operating the set of hand massagers (20) may or may not operate. One or both of the set of massagers may operate at a time.

Accordingly, the attached to the backside of the hand covering (12) may be a control mechanism (18). The control mechanism (18) may control the functions and intensity of the set of finger massagers (16) and the set of hand massagers (20). The control mechanism (18) may be internally housed within the hand covering (12) with a set of control buttons exposed outside the hand covering (12). Alternatively, the control mechanism (18) may be attached above the hand covering (12). The control mechanism (18) may be battery operated to enable the user to easily transport and store the heated massage glove (10).

Within the hand covering (12) and running through the set of finger segments (14) may be a heating apparatus. The heating apparatus may include a plurality of heating wires or coils which run throughout the hand covering (12). The heating apparatus is controlled by the control mechanism (18), wherein the control mechanism (18) operates the on/off functions of the heating apparatus as well as the intensity. The heating apparatus may have more intense heating sections concentrated around the set of finger massagers (16) and the set of hand massagers (20) to provide additional relief to the user's fingers and knuckles.

Finally, wrapping around a wrist portion of the hand covering (12) may be an adjustment strap (22). The adjustment strap (22) may be an elastic strap that fastens with a snap, a hook, a hook and loop fastener or the like. The adjustment strap (22) secures the hand covering (12) around the user's wrist to ensure that the heated massage glove (10) is not removed during use. Additionally, the adjustment strap (22) may include a set of wrist massagers and a set of heated wires housed within the wrist portion to sooth any aches and pains the user may experience in their wrist. Therefore, the heated massage gloves (10) may provide relief to multiple portions of the user's hand so that concentrated and customized relief is given to the user.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A heated massage glove comprising:
a hand covering to envelope a hand of a user;
a set of finger segments to accept the fingers of said user;
a set of finger massagers attached within said set of finger segments, where said set of finger massagers vibrate and pulse against said fingers;
a set of hand massagers attached to a backside of said hand covering, wherein said set of hand massagers vibrate and pulse against a backside of said hand;
a heating apparatus housed within said hand covering, where said heating apparatus includes a plurality of wires to heat said hand covering; and
a control mechanism attached to said backside of said hand covering wherein said control mechanism controls how intense said set of finger massagers and said set of hand massagers vibrates and pulses and how said heating apparatus heats.

2. The heated massage glove of claim 1, wherein said control mechanism includes a set of buttons exposed outside of said hand covering.

3. The heated massage glove of claim 1, wherein said control mechanism is battery operated.

4. The heated massage glove of claim 1, wherein said heated massage glove is available as a pair of heated massage gloves.

5. The heated massage glove of claim 1, wherein said hand covering includes an adjustment strap wrapping around a wrist portion of said hand of said user to secure said heated massage glove around said hand.

6. The heated massage glove of claim 5, wherein said adjustment strap is secured with a hook and loop fastener.

7. The heated massage glove of claim 5, wherein said adjustment strap houses a set of wrist massagers.

8. The heated massage glove of claim 1, wherein said plurality of wires included with said heating apparatus are concentrated around said set of finger massagers and set of hand massagers.

9. The heated massage glove of claim 1, wherein said set of finger massagers operates independent of set of hand massagers.
